(19) United States
(12) Patent Application Publication
(10) Pub. No.: US 2011/0137188 A1
(43) Pub. Date: Jun. 9, 2011

(54) SLEEP ASSISTANT SYSTEM

(75) Inventors: Bo-Jau KUO, Taipei City (TW); Ching-Hsiu YANG, Taipei City (TW)

(73) Assignee: NATIONAL YANG-MING UNIVERSITY, Taipei City (TW)

(21) Appl. No.: 12/688,451

(22) Filed: Jan. 15, 2010

(30) Foreign Application Priority Data
Dec. 3, 2009 (TW) 098141297

(51) Int. Cl. A61B 5/0402; A61B 5/11

(52) U.S. Cl. 600/509; 600/595

(57) ABSTRACT

The present invention relates to a system for sleeping assistance which comprises an electrocardiogram collector, a heartbeat recognition device, a frequency domain analysis device and a nerve active judgment device. Frequency spectrum and heartbeat rate analysis are made by the measured electrocardiogram to find out what nerve actives effect sleeping and to give corresponding feedback.

### FIELD OF THE INVENTION

The present invention relates to a system for diagnosis nervous active in order to assist sleep, and a method for using the same.

### BACKGROUND OF THE INVENTION

1. The Development of Non-Invasive Diagnostics Technology

The progress of medical science is much advanced than ever and can be classified structurally and functionally. With respect to functionally, Various methods for determine every parts of human body's organs and tissues were developed by physiologist. For hundreds years, it always has a corresponding way to measure and diagnosis function of every organ. Exploration was the principle considerations in the former methods, it emphasized the precision on signal measurement. To achieve this objective, invasive tools and techniques were often used, such as use a catheter to extend into heart through arteries in order to practice cardiac catheterization, and these kind of skills not only suffering pain but also dangerous and neglect receiver's feelings. Now, the gradually formed of another concept for the ultimately development of invasive techniques is non-invasive diagnostics technology. Non-invasive technology use painless and unharmful tools and techniques for the functional measurement and diagnosis of body's organs as opposed to pain results from invasive methods. Previously, accuracy and usefulness requirements were only an ideology and not always satisfied because of invasive methods unable to invasive into body to obtain precise physiologic events. Recently, because of drastically advanced in signal probing and data management skills, especially the achievement of software engineering, we can compensate the vulnerability of non-invasive methods by the powerful computer's operation. Non-invasive diagnostics technology can represent by heart rate variability (HRV; Anonymous 1996). HRV analysis can obtain a quantitative profile of autonomic nervous function by electrocardiography of electrode detection on body surface through tediously digital signal treatment. Inventors have found its functions and use these skills to diagnosed the depth of anesthesia (Yang et al. 1996), brain death (Kuo et al. 1997), prognosis of critical illness (Yien et al. 1997), senescence (Kuo et al. 1999), gender difference (Kuo et al. 1999) or its related diseases. In view of receiver's convenience and comfort, there still have a broad space for the development of non-invasive methods.

Furthermore, triaxial acceleration test is an important indicator of body activity, its measurement does not need to contact body directly. Electrocardiography and body activities are two kinds of important physiological signals to be detected and trying to be combine in sensors designation, it will increase credibility and convenience in quantify the change of various physiological signals for clinical (healthy people or patients) and research (in animal physiology and behavior).

2. The Relationship Between Heart Rate to Autonomic Nervous System and Motility to Sleep

Many research studies noted that low heart rate and sympathetic nervous activities in sleep will in company with high parasympathetic nervous activities, and on the contrary when awake (Yang et al. 2002; Yang et al. 2003). Further studies point out low heart rate and high parasympathetic nervous activities will occur just prior to going to sleep (Kuo et al. 2007). However, disrupted sleep and awake from sleep has been associated with early elevated heart rate and sympathetic nervous activities. Many sleep disorder patients are often associate with high heart rate and sympathetic activities and/or low parasympathetic functions when insomnia. It appears that heart rate and autonomic nervous function have some relationship with sleep disorders like unable sleep or vigilant sleep. Therefore, methodologically and theoretically, decrease heart rate and sympathetic nervous activities and/or increase parasympathetic nervous activities shall facilitate sleep effectively. Heart rate and autonomic nervous function are an ultimate expression of various factors of stresses, psychological and physiological problems. High heart rate and sympathetic activities with a low parasympathetic activities is a reliable psychological and physiological index for the use of sleeping aid before sleep.

Calculated motility obtain from triaxial accelerator can be linear and quick response body motility, which is used widely for many kinds of purposes, and we found that the variation of its parameters can prompt response sleep and wake activities precisely in our studies. Quantify the magnitude of activities during sleeping is an alternative physiological index, for example, very low total activities in sleeping while high in disrupted sleeping.

3. Feedback Design

Until today, we already can easily collect, quantify and analyse physiological signals with accuracy, besides, more reliable wireless transmission and reception of message plus high accuracy of advanced computer operation bring about the gradually maturity for design a real time detection and feedback of physiological signals control. Recently, this can be complete precisely by inventors with a real time control switch of brain wave frequencies. The feasibility of various integration and assorted design to promote feedback designs can also be expected.

### SUMMARY OF THE INVENTION

Recent years, we can obtain an exactly quantitative values of body heart rate and autonomic nervous activities by making use of electrocardio- and triaxial accelerator's signals after digital signal processing because of the continuous breakthrough and innovation of digital diagnosis techniques. However, in medical researches the change of these physiological parameters relating to sleepless and/or wake situation. Moreover, these physiological signals can be detected by non-invasive methods and its physiological signals can be wireless transmitted as long as it contact with skin even for a long-term period. With a simple design, it is conducive to sleep let alone sleep disturbance for reach a real sleep-aid effect.

### BRIEF DESCRIPTION OF THE FIGURES

FIG. 1: shows a system of the present invention in one embodiment, which schematically illustrates a structure comprises an electrocardiogram collector and an acceleration sensor; it is formed necklace-like shape for convenience of wear.

FIG. 2: shows a system of the present invention in one embodiment, which is an block schematic diagram of an electrocardiogram collector.

FIG. 3: is a schematic diagram of evaluation results of the present invention, which is obtained from a variety of data such as heart rate, quantitative autonomic nervous information of heart and total motility signals; and provide a proper feedback reaction when a difficulty sleeping patient is determined. Subscript indicates relative change for each signal on awake and start or turn-off time of the feedback device.

FIG. 4: shows a flow chart of biofeedback control of the present invention.

### DETAILED DESCRIPTION OF THE INVENTION

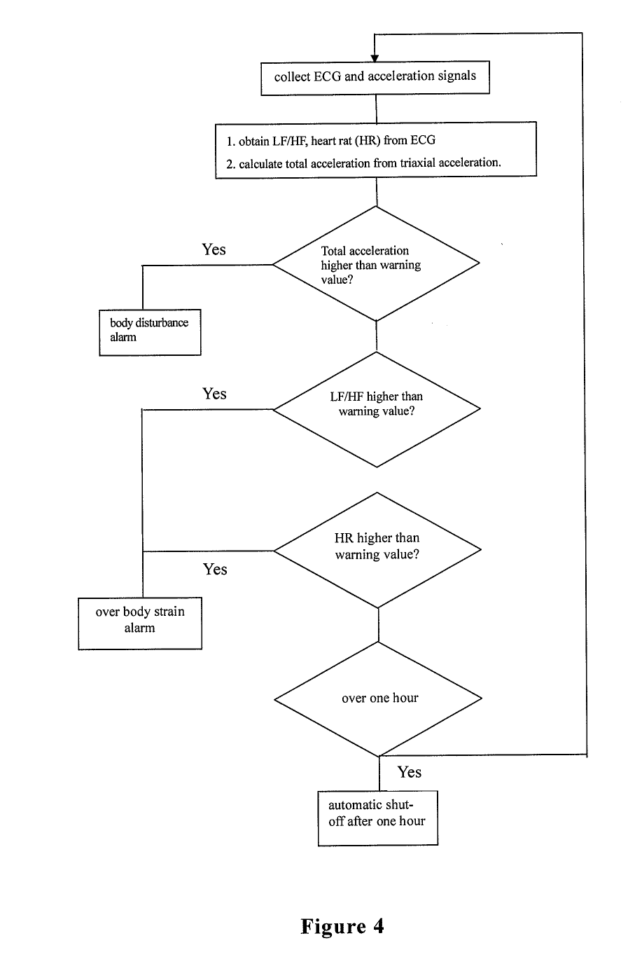

For purposes of foregoing invention, a sleep assistant system which comprises an electrocardiogram collector for detect and record electrocardio-signals is provided by the the present invention. In one preferred embodiment, said electrocardiogram collector includes a detect electrode and a reference electrode for attaching on the skin surface in non-invasive way; a heartbeat recognition device, which obtains cardiac cycle sequences from electrocardio-signals collected by said electrocardiogram collector through peck detection procedure or other method to cooperate with noise separation procedure; a frequency domain analysis device, which includes (but not limit to) sampling the heart cycle, Hamming operation (for prevent heart cycle leak each other of individual frequency component in sampling), fast Fourier transform to transfer sampling results of heart cycle into power density spectrum, and calculates the power of low frequency (0.04˜0.15 Hz) and high frequency (0.15˜0.4 Hz) from said power density spectrum by integral quantification to obtain total power and low-high frequency power ratio; finally, determine whether cardiac cycle sequences and heart sympathetic nervous activities represent by low-high power ratio are higher than warning value for the diagnosis of sleep disrupted effect and propose corresponding counter measure(e.g. relax and relieve stress) and feedback (automatic shut-off). Simple process shows in FIG. 4.

In summary, the present invention provides a sleep assistant system, which comprises:

(a) an electrocardiogram collector;

(b) a heartbeat recognition device, which obtains cardiac cycle sequences from electrocardio-signals collected by said electrocardiogram collector;

(c) a frequency domain analysis device, which obtains power of different frequency from said cardiac cycle sequences; and

(d) a nerve active judgment device, which determines whether said cardiac cycle sequences and said power of different frequency have any effect on disrupted sleep.

Since body activity is another factor which influence sleep, in a preferred embodiment, the present system further comprises:

(a) an acceleration sensor;

(b) a motility computing device, which computes body motilities from said accelerator's signals of acceleration sensor; and

(c) a hyperergasia judging device, which interprets said computed body motilities and estimates its sleep disrupted effect.

Said acceleration sensor preferably is an acceleration induction integrated circuit. Said motility computing device measures acceleration to obtain vector sum of triaxial directions(x, y, z) and computes body motilities for the diagnosis of sleep disrupted effect and propose corresponding counter measure and feedback.

In order to improve portability and remote medical care services of the system, in one preferred embodiment, the present invention also comprises a wireless transceiver and a remote data processing device, wherein said wireless transceiver responsible for transmit recorded electrocardio-signals of said electrocardiogram collector or recorded acceleration signals of said acceleration sensor to said remote data processing device, or receive data of said remote data processing device; Said remote data processing device responsible for receive and analyse said recorded signals and return results to said wireless transceiver or to health agencies. The transport interface use by said wireless transceiver and remote data processing device are radio system, wireless network, infra-red, bluetooth, wireless radio-frequency, GSM, PHS and CDMA, or any wireless protocol.

The invention also provides a method of sleep states estimation, which includes measuring said cardiac cycle sequences; analysing spectrum of said cardiac cycle sequences; and estimating nervous activities from the results of said cardiac cycle sequences or said spectral analysis. Nerve described herein includes, but not limit to, sympathetic nerve and parasympathetic nerve; and in a further preferred embodiment, the present method comprises measuring body mortilities for sleep states judgment.

### EXAMPLES

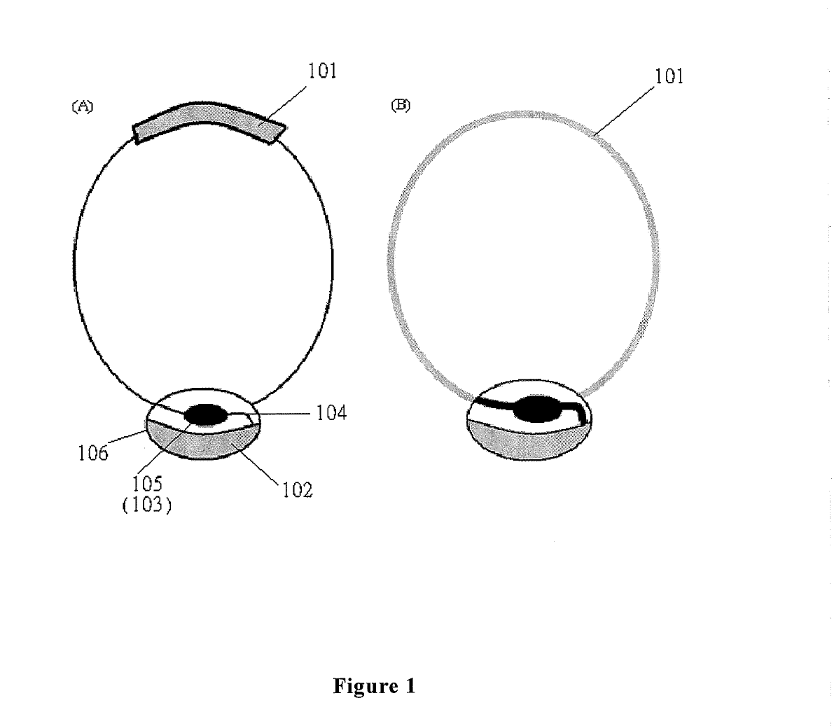

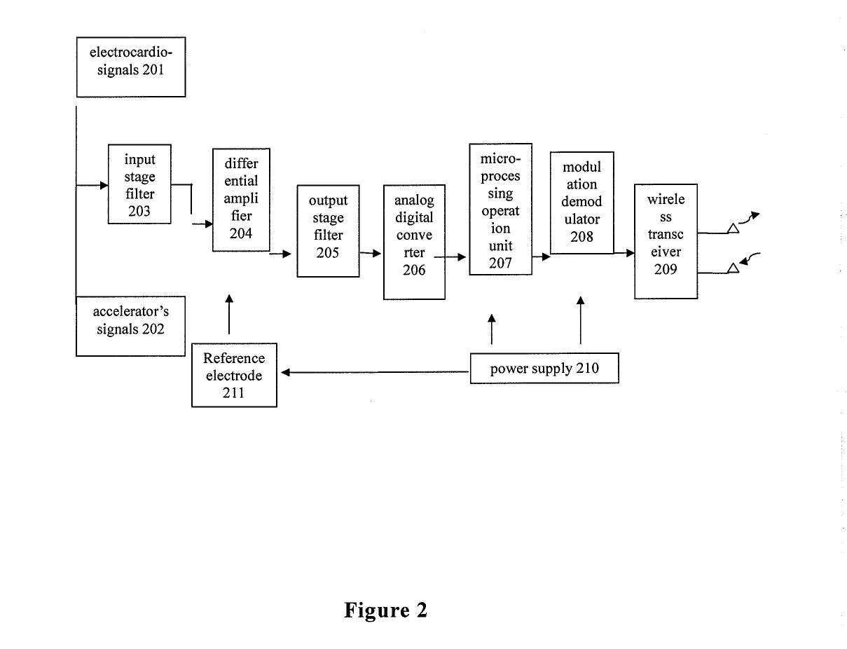

The electrocardiogram collector of present system is composed of two electrodes. These individual components can be integral each other by a necklace-like conductor as illustrated in FIG. 1; said sensor can be wear around the neck with the reference electrode 101 located on posterior cervical region (FIG. 1A), its neck contacting region may be a metal sheet or consider the most part of the necklace-like conductor as a reference electrode (FIG. 1B), while the heart wave and body temperature input electrode 102 is located on the chest. The necklace-like design not only can hold electrodes in place so that not to loss but also provide a excellent contact between electrode with neck and chest. Mini-amplifier 103, radio transmitted circuit 104 and battery 105 can be designed on pendants. The layout architecture of electrocardiogram collector 106 shows in FIG. 2, where said electrocardio- 201 and accelerator's signals 202 are processed sequentially through input stage filter 203, differential amplifier 204, output stage filter 205, analog digital converter 206, micro-processing operation unit 207, modulation demodulator 208, and eventually emit remote signals by said wireless transceiver 209.

Basic circuit which collect electrical signals consists of electrodes on both sides, this electrocardiogram collector adopts two-electrode input method for simplify usage and increase reliability although two-electrode input method has more serious noise disturbance than three-electrode differential input method, but it can be overcome by proper filter circuit and optical isolator circuit. Present embodiment adopts prior amplifier circuit of preceding patent (Kuo 1999) to amplify input electrocardio-signals of said two-electrode, for example, and obtains a useful wave form of signal-to-noise ratio. Since necklace-like heart wave and body temperature signals may intermittent due to body movement, therefore said electrodes need to be fixed at least 5 minutes for steady signals. Alternatively, a specific method for handing said noise is used.

Perform following procedures for processing digital electrocardiogram and impulse signals (Kuo et al. 1999; Yang et al. 2000). First, find the peck wave of every heart beat with peck detection procedure (Kuo and Chan 1992) to represent each heart signal. Measure parameters such as altitude and duration or the like from each heart signal peck, and calculate the average and standard deviation of each parameter as standard template. Uses said template for comparison in subsequent heart signals, if comparison results of a heart signal is fall outside three standard deviation of said template then it will be regarded as a noise to be delete. Next, measures interval of time between two adjacent peck of heart signals as its heart cycle. Calculate the average and standard deviation of all heart cycle, and then confirm every heart cycles, if certain heart cycles fall outside three standard deviation then it will also be regarded as noise or unstable signal and to be filter out. Cardiac cycle sequences obtain by this recognition procedure will be processed in subsequent analysis.

All conformed heart cycle sequences will be under sampling and value-preserved procedure with 7.11 Hz in order to maintain time coherence, and utilize Fourier transform for spectrum analysis. At first, eliminate signal linear drift for prevent of interference of low frequency band, adopt Hamming operation as well in order to prevent leak each other of individual frequency component in spectrum (Kuo 1999; Kuo and Chan 1993). Subsequently, take data of 288 seconds (2048 points) for fast Fourier transform (Cooley and Turkey 1965) to obtain power density spectrum , and compensate the effect result from sampling and Hamming operation (Kuo 1999; Kuo et al. 1999).

Power density spectrum of heart rate variability (HRV) integrally quantify two power frequencies therein includes power of low-frequency(LF, 0.0˜40.15 Hz) and high-frequency (HF, 0.1˜50.4 Hz). Total power (TP), equalizing parameter of low- to high-frequency ratio (LF/HF) is obtained concurrently (Anonymous 1996; Kuo et al. 1999; Yang et al. 2000). These parameters will attain normal distribution through log-transformation (Kuo et al. 1999).

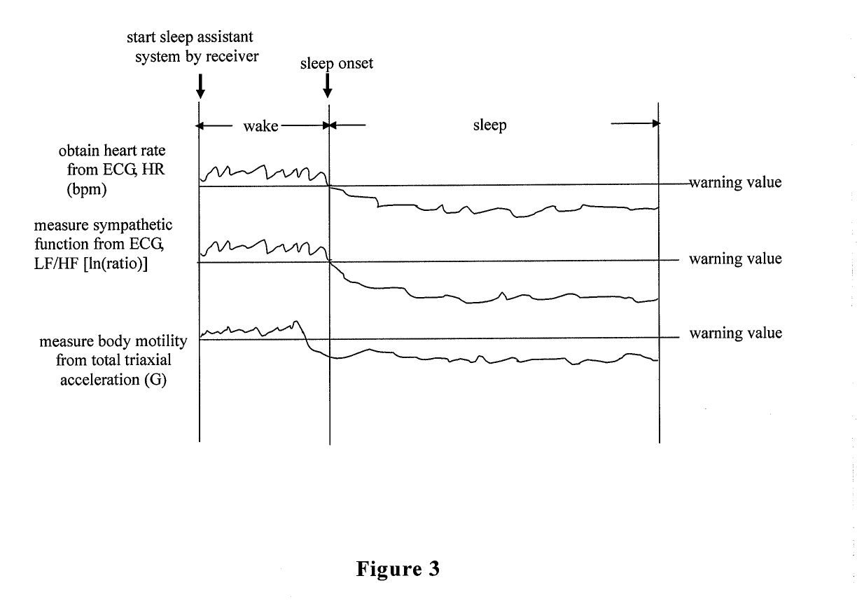

HF can be viewed as an index of heart parasympathetic activities and LF/HF can be viewed as an index of heart sympathetic activities in experiment results according to the experience of present inventors (Kuo et al. 1999; Kuo et al. 1997; Yang et al. 2000; Yien et al. 1997) and the consensus of European and American physician (Anonymous 1996). The relationship between electrocardiogram and sleep shows in FIG. 3.

Sensor may has an acceleration sensory integrated circuit(or collect by other method), which measures acceleration of triaxial directions(x, y, z) and integrates acceleration of triaxial directions into one signal to obtain gross acceleration.

Gross acceleration can be expressed as \sqrt{x²+y²+z²}, and use this signal represents patient's motility.

### DESCRIPTION OF REFERENCE NUMBER

101 reference electrode
102 input electrode
103 mini-amplifier
104 radio transmitted circuit
105 battery
106 electrocardiogram collector
201 electrocardio-signals
202 accelerator's signals
203 input stage filter
204 differential amplifier
205 output stage filter
206 analog digital converter
207 micro-processing operation unit
208 modulation demodulator
209 wireless transceiver
210 power supply
211 reference electrode

1. A sleep assistant system, which comprises:
(a) an electrocardiogram collector;
(b) a heartbeat recognition device, which obtains cardiac cycle sequences from electrocardio-signals collected by said electrocardiogram collector;
(c) a frequency domain analysis device, which obtains power of different frequency from said cardiac cycle sequences; and
(d) a nerve active judgment device, which determines whether said cardiac cycle sequences and said power of different frequency have any effect on disrupted sleep.

2. The sleep assistant system of claim 1, wherein said electrocardiogram collector includes a detect electrode and a reference electrode for attaching on the skin surface.

3. The sleep assistant system of claim 1, wherein said heartbeat recognition device includes a peck detection procedure and a noise separation procedure.

4. The sleep assistant system of claim 1, wherein frequency domain analysis device comprises:
(a) sampling the heart cycle;
(b) Hamming operation for prevent heart cycle leak each other of individual frequency component in sampling;
(c) fast Fourier transform to transfer sampling results of heart cycle into power density spectrum; and
(d) calculates the power of low frequency (0.04˜0.15 Hz) and high frequency(0.15˜0.4 Hz) from said power density spectrum by integral quantification to obtain total power and low-high frequency power ratio.

5. The sleep assistant system of claim 1, wherein said nerve active judgment device includes:
determine whether said cardiac cycle sequences or said low-high power ratio are higher than warning value for the diagnosis of sleep disrupted effect.

6. The sleep assistant system of claim 1, which further includes an automatic shut-off element.

7. The sleep assistant system of claim 1, which further comprises:
(a) an acceleration sensor;
(b) a motility computing device, which computes body motilities from said accelerator's signals of acceleration sensor; and
(c) a hyperergasia judging device, which interprets said computed body motilities and estimates its sleep disrupted effect.

8. The sleep assistant system of claim 7, wherein said acceleration sensor is an acceleration induction integrated circuit.

9. The sleep assistant system of claim 7, wherein said motility computing device measures acceleration to obtain vector sum of triaxial directions (x, y, z).

10. The sleep assistant system of claim 1, which further comprises a wireless transceiver and a remote data processing device, wherein
wherein said wireless transceiver responsible for transmit recorded electrocardio-signals of said electrocardiogram collector or recorded acceleration signals of said acceleration sensor to said remote data processing device, or receive data of said remote data processing device; said remote data processing device responsible for receive and analyse said recorded signals and return results to said wireless transceiver or to health agencies.

11. A sleep states estimation method, which includes:
(a) measuring said cardiac cycle sequences;
(b) analysing spectrum of said cardiac cycle sequences; and
(c) estimating nervous activities from the results of said cardiac cycle sequences or said spectral analysis.

12. The sleep states estimation method of claim 11, wherein said nerve is sympathetic nerve or parasympathetic nerve.

13. The sleep states estimation method of claim 11, which comprises:
(a) measuring said body motilities;
(b) judging said body motilities have any effect to said disrupted sleep.

14. The sleep assistant system of claim 7, which further comprises a wireless transceiver and a remote data processing device, wherein
wherein said wireless transceiver responsible for transmit recorded electrocardio-signals of said electrocardiogram collector or recorded acceleration signals of said acceleration sensor to said remote data processing device, or receive data of said remote data processing device; said remote data processing device responsible for receive and analyse said recorded signals and return results to said wireless transceiver or to health agencies.
